(19) United States
(12) Patent Application Publication
(10) Pub. No.: US 2023/0026895 A1
(43) Pub. Date: Jan. 26, 2023

(54) METHOD AND SYSTEM FOR MANAGING DENTAL ARTICLE INFORMATION BASED ON UHF RFID

(71) Applicants: KONG TECH Co., Ltd., Seongnam-si (KR); OSSTEMIMPLANT CO., LTD., Seoul (KR); KOREA DESIGN SCIENCE INSTITUTE, Seoul (KR)

(72) Inventors: Dong Un LEE, Seoul (KR); Hak Kyung LEE, Sungnam-si (KR); Ho Jin LIM, Seoul (KR); Myoung Pyo HONG, Hwaseong-si (KR); Young Soo JUNG, Seoul (KR); Sang Yeon PARK, Hanam-si (KR); Young Kyu CHI, Seoul (KR); Yong Jin KIM, Gimpo-si (KR); Gyu Nam KIM, Goyang-si (KR)

(73) Assignees: KONG TECH Co., Ltd., Seongnam-si (KR); OSSTEMIMPLANT CO., LTD., Seoul (KR); KOREA DESIGN SCIENCE INSTITUTE, Seoul (KR)

(21) Appl. No.: 17/592,507

(22) Filed: Feb. 3, 2022

(30) Foreign Application Priority Data
Jul. 22, 2021 (KR) 10-2021-0096408

(51) Int. Cl. G16H 40/20; A61B 50/10; A61C 8/00

(52) U.S. Cl. CPC G16H 40/20; A61B 50/10; A61C 8/0089; A61C 2204/005; A61B 2050/105

(57) ABSTRACT

The present invention relates to a method and a system for managing dental article information based on a UHF RFID, and more particularly, to a method and a system for managing dental article information based on a UHF RFID, capable of automatically monitoring a location, a stock, and an expiration date of an article used by medical staffs of a dental clinic without an additional action of the medical staff, and providing information and a notification related to a dental article to the medical staffs of the dental clinic.

### CROSS-REFERENCE TO RELATED APPLICATIONS

The present application claims the benefit of priority under 35 U.S.C. § 119 to Korean Patent Application No. 10-2021-0096408, filed on Jul. 22, 2021, in the Korean Intellectual Property Office, the entire contents of which is hereby incorporated by reference.

### BACKGROUND OF THE INVENTION

### 1. Field of the Invention

The present invention relates to a method and a system for managing dental article information based on a UHF RFID, and more particularly, to a method and a system for managing dental article information based on a UHF RFID, capable of automatically monitoring a location, a stock, and an expiration date of an article used by medical staffs of a dental clinic without an additional action of the medical staff, and providing information and a notification related to a dental article to the medical staffs of the dental clinic.

### 2. Description of the Related Art

There are various types of dental articles, and a suitable dental article (e.g., an implant fixture, etc.) has to be used according to each operation and each condition of a patient.

In addition, since the dental article has an expiration date, it is difficult to hold a large number of stocks, so that there is a difficulty in managing a proper amount of dental articles by a work such as bookkeeping for the dental articles through a handwritten ledger or the Excel in a hospital.

Meanwhile, Related patent 1 (Korean Patent Registration No. 10-1866898) discloses a method including: printing general information corresponding to an article and assigning an RFID tag having a specific serial number for each predetermined area of a label base roll; attaching an RFID tag to a rear surface of a main trademark or a highlighting mark prepared to be attached to the article; cutting the label base roll to which the RFID tag is assigned into individual unit labels having a predetermined size; attaching the individual unit label obtained through the cutting and the main trademark or the highlighting mark having the rear surface to which the RFID tag is attached to predetermined locations of the article, respectively; reading information from the RFID tag attached to the label, the main trademark, or the highlighting mark of the article by an RFID reader when the article is put into/released from a warehouse, a distribution center, or a shop; and transmitting the information read by the RFID reader to a computer system, and tracking and managing the article by processing the information by the computer system.

However, Related patent 1 described above has to input the tag of the article to the RFID reader similarly to barcode scanning, so that an additional action of a medical staff is involved, and a function required in dental clinics and hospitals, such as stock management for dental articles, may not be performed.

### Documents of Related Art

### Patent Documents

(Patent document 0001) Korean Patent Registration No. 10-1866898 (Registration date: Jun. 5, 2018)

### SUMMARY OF THE INVENTION

An object of the present invention is to provide a method and a system for managing dental article information based on a UHF RFID, capable of automatically monitoring a location, a stock, and an expiration date of an article used by medical staffs of a dental clinic without an additional action of the medical staff, and providing information and a notification related to a dental article to the medical staffs of the dental clinic.

To achieve the objects described above, according to the present invention, there is provided a system for managing dental article information, the system including: a UHF RFID tag attached to a dental article; a storage module having a space in which the dental article is stored; a UHF RFID reader disposed on an upper side of the storage module, and configured to read reading article information of the UHF RFID tag attached to the dental article stored in the storage module; and a server system configured to receive the reading article information, and store location information of the UHF RFID reader and detailed article information corresponding to the reading article information, wherein the server system is configured to perform: a stock information provision step of deriving current stock information for each dental article for each of a consultation room, a disinfection room, and an operating room based on the location information of the UHF RFID reader and the reading article information, and providing the current stock information to a user terminal; a notification information provision step of setting safety stock information and expiration date information for each dental article based on information input from the user terminal, and generating notification information based on the current stock information, the safety stock information, and the expiration date information to provide the notification information to the user terminal; and a stock management step of providing the current stock information and the safety stock information for each dental article to the user terminal, and providing an order interface for each dental article to a user.

In some embodiments of the present invention, the storage module may include: a cabinet having a single inner reception space without an inner partition wall; and a system box stacked inside the cabinet, and in which at least one dental article is stored, an antenna module of the UHF RFID reader may be arranged in a planar shape on a top surface of the cabinet, and the antenna module of the UHF RFID reader and a top surface of the inner reception space of the cabinet may be spaced apart from each other by 20 cm or more.

In some embodiments of the present invention, the UHF RFID reader may be configured to identify UUID information of the UHF RFID tag when the UHF RFID tag is present inside the storage even when the user does not bring the UHF RFID tag close to the UHF RFID reader, and the UHF RFID reader may be configured to transmit information of a plurality of UHF RFID tags that are present inside the storage module to the server system.

In some embodiments of the present invention, the detailed article information may be stored in the server system for each individual dental article, and the detailed article information may include article name information of the dental article and the expiration date information of the dental article.

In some embodiments of the present invention, a screen of the user terminal displayed by the stock information provision step may include: a space selection layer for selecting one of the consultation room, the disinfection room, and the operating room; and a stock information layer for displaying the current stock information of one of the consultation room, the disinfection room, and the operating room according to a space selected in the space selection layer.

In some embodiments of the present invention, the stock information provision step may include: deriving information on a type and an amount of a dental article that is present inside the storage module at a preset interval based on information received from the UHF RFID reader; and deriving, when a fluctuation occurs in the amount of the dental article that is present inside the storage module, usage information of the dental article based on the fluctuation, and the stock information layer may display: stock information of the dental article, which is sorted by a stock amount; and a specific dental article extracted by applying a preset criterion to the usage information.

In some embodiments of the present invention, the notification information provision step may include: a safety stock setting step of providing an interface for setting a notification state for a safety stock and a safety stock amount for each dental article in the user terminal; a safety stock notification derivation step of deriving safety stock notification information for a dental article in which a current stock amount for each dental article derived based on the current stock information is less than the safety stock amount; and a list provision step of providing the safety stock notification information in a form of a list together with time information related to the safety stock notification information.

In some embodiments of the present invention, the detailed article information may include the expiration date information for each dental article, and the notification information provision step may include: an expiration date setting step of providing an interface for setting a reference expiration date for all dental articles in the user terminal; an expiration date notification derivation step of deriving expiration date notification information for a dental article in which an expiration date has reached the reference expiration date among the dental articles; and a list provision step of providing the expiration date notification information in a form list together with time information related to the expiration date notification information.

To achieve the objects described above, according to the present invention, there is provided a method for managing dental article information by using a dental article management system, in which the dental article management system includes a UHF RFID tag attached to a dental article, a storage module having a space in which the dental article is stored, a UHF RFID reader disposed on an upper side of the storage module, and configured to read reading article information of the UHF RFID tag attached to the dental article stored in the storage module, and a server system configured to receive the reading article information, and store location information of the UHF RFID reader and detailed article information corresponding to the reading article information, the method comprising: a stock information provision step of deriving, by the server system, current stock information for each dental article for each of a consultation room, a disinfection room, and an operating room based on the location information of the UHF RFID reader and the reading article information, and providing the current stock information to a user terminal; a notification information provision step of setting, by the server system, safety stock information and expiration date information for each dental article based on information input from the user terminal, and generating notification information based on the current stock information, the safety stock information, and the expiration date information to provide the notification information to the user terminal; and a stock management step of providing, by the server system, the current stock information and the safety stock information for each dental article to the user terminal, and providing an order interface for each dental article to a user.

According to one embodiment of the present invention, the location, the stock, and the expiration date of the article used by the medical staffs of the dental clinic can be automatically monitored without the additional action of the medical staff.

According to one embodiment of the present invention, the information and the notification related to the dental article can be provided to the medical staffs of the dental clinic.

### BRIEF DESCRIPTION OF THE DRAWINGS

FIG. 1 schematically shows an overall configuration of a system for managing dental article information according to one embodiment of the present invention.

FIG. 2 schematically shows a structure in which a UHF RFID reader is coupled to a storage module according to one embodiment of the present invention.

FIG. 3 schematically shows an operation and an internal configuration of a server system of the system for managing the dental article information according to one embodiment of the present invention.

FIG. 4 schematically shows an operation of a stock information provision unit according to one embodiment of the present invention.

FIG. 5 schematically shows a screen of a user terminal displayed by a stock information provision step according to one embodiment of the present invention.

FIG. 6 schematically shows detailed processes of a notification information provision step according to one embodiment of the present invention.

FIG. 7 schematically shows a screen of the user terminal for performing setting on a safety stock in the notification information provision step according to one embodiment of the present invention.

FIGS. 8A-8C schematically show an automatic update process for the safety stock in the notification information provision step according to one embodiment of the present invention.

FIG. 9 schematically shows a screen of the user terminal for providing a notification list in the notification information provision step according to one embodiment of the present invention.

FIG. 10 schematically shows detailed processes of the notification information provision step according to one embodiment of the present invention.

FIG. 11 schematically shows a screen of the user terminal for performing setting on an expiration date in the notification information provision step according to one embodiment of the present invention.

FIG. 12 schematically shows a screen of the user terminal in a stock management step according to one embodiment of the present invention.

FIG. 13 schematically shows a screen of the user terminal in a comprehensive information provision step according to one embodiment of the present invention.

FIG. 14 schematically shows an internal configuration of a computing system such as the server system according to one embodiment of the present invention.

### DETAILED DESCRIPTION OF THE INVENTION

Hereinafter, various embodiments and/or aspects will be disclosed with reference to the drawings. In the following description, for the purpose of explanation, numerous specific details are set forth in order to provide a thorough understanding of one or more aspects. However, it shall also be appreciated by a person having ordinary skill in the art to which the present invention pertains that such aspect(s) may be practiced without the specific details. The following description and the accompanying drawings will be set forth in detail for specific illustrative aspects among the one or more aspects. However, the aspects are provided for illustrative purposes, some of various schemes based on principles of various aspects may be employed, and descriptions set forth herein are intended to include all the various aspects and equivalents thereof.

In addition, various aspects and features will be proposed by a system that may include a plurality of devices, components, and/or modules. It will also be understood and appreciated that various systems may include additional devices, components, and/or modules, and/or that various systems may not include all of the devices, components, modules, and the like discussed in association with the drawings.

The terms “embodiment”, “example”, “aspect”, “illustration”, and the like used herein may not be construed as indicating that an aspect or design set forth herein is preferable or advantageous over other aspects or designs. Each of the terms “unit”, “component”, “module”, “system”, “interface”, and the like used below generally represents a computer-related entity, and may represent, for example, hardware, a combination of hardware and software, or software.

In addition, it is to be understood that the terms “include” and/or “comprise” indicate the presence of corresponding features and/or elements, but do not preclude the presence or addition of one or more other features, elements, and/or groups thereof.

In addition, although the terms including an ordinal number such as “first” or “second” may be used to describe various elements, the elements are not limited by the terms. The above terms are used merely for the purpose of distinguishing one element from another element. For example, a first element may be termed as a second element, and similarly, a second element may also be termed as a first element without departing from the scope of the present invention. The term “and/or” includes any combination of a plurality of described relevant items, or one of the described relevant items.

In addition, unless defined otherwise, all terms used in embodiments of the present invention, including technical and scientific terms, have the same meaning as those commonly understood by a person having ordinary skill in the art to which the present invention pertains. Any terms as those defined in generally used dictionaries are to be interpreted to have the meanings equal to the contextual meanings in the relevant field of art, and are not to be interpreted to have idealistic or excessively formalistic meanings unless explicitly defined in the embodiments of the present invention.

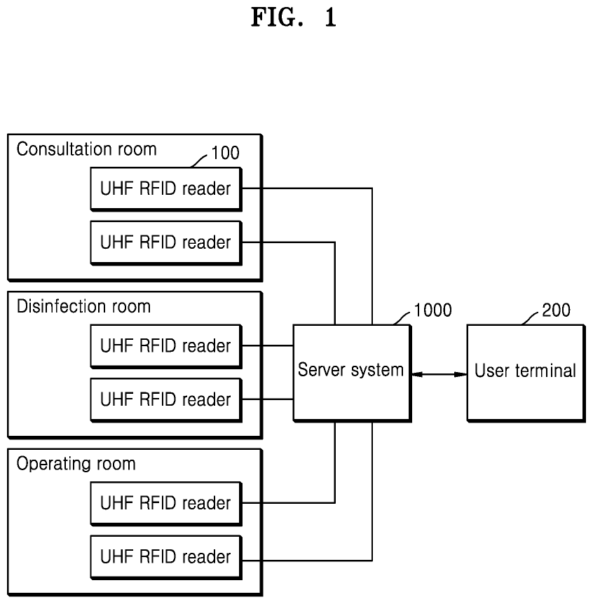

FIG. 1 schematically shows an overall configuration of a system for managing dental article information according to one embodiment of the present invention.

According to embodiments of the present invention, a system for managing dental article information may include: a UHF RFID tag attached to a dental article; a storage module having a space in which the dental article is stored; a UHF RFID reader disposed on an upper side of the storage module, and configured to read reading article information of the UHF RFID tag attached to the dental article stored in the storage module; and a server system configured to receive the reading article information, and store location information of the UHF RFID reader and detailed article information corresponding to the reading article information.

A dental clinic may be divided into a consultation room, a disinfection room, and an operating room, and at least one UHF RFID reader may be provided in a storage module of each of the rooms. Meanwhile, a UHF RFID tag may be attached to a dental article, and the UHF RFID reader may identify the dental article that is present in the storage module.

In such a state, medical staffs in the dental clinic may perform their tasks as they normally do without an additional action such as an action of bringing the tag close to the RFID reader, and the UHF RFID reader may collect information on the dental article that is present in the storage module to transmit the collected information to the server system.

Thereafter, the user terminal may inquire where a specific article is basically located through the server system, and may easily recognize stock information, notification information, and the like, which will be described below.

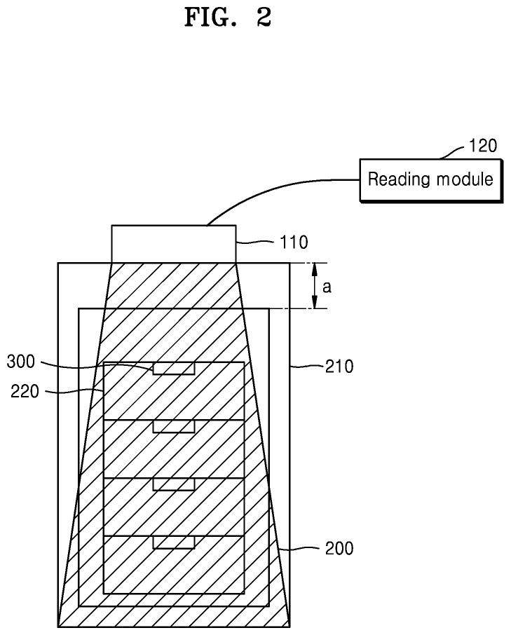

FIG. 2 schematically shows a structure in which a UHF RFID reader is coupled to a storage module according to one embodiment of the present invention.

According to one embodiment of the present invention, the storage module may include: a cabinet having a single inner reception space without an inner partition wall; and a system box stacked inside the cabinet, and in which at least one dental article is stored.

A front door may or may not be installed in the cabinet.

Meanwhile, it may be preferable that a separate partition wall such as a shelf does not exist inside the cabinet. This is because, due to characteristics of the UHF RFID, there may be an error in recognition of a UHF RFID tag located in a space far away from an antenna module of the UHF RFID reader when a horizontal partition wall such as a shelf is present.

Therefore, the dental article may be preferably stored and managed by stacking the system box in the inner reception space of the cabinet.

Meanwhile, it may be preferable that the antenna module of the UHF RFID reader is arranged in a planar shape on a top surface of the cabinet, and the antenna module of the UHF RFID reader and a top surface of the inner reception space of the cabinet are spaced apart from each other by 20 cm or more.

Since the antenna module has a planar shape, UHF RFID tags located in a lower region may be recognized more uniformly. However, since the antenna module has a limited area, it may be difficult to recognize a tag that is present in a location directly under the antenna module while deviating from the area of the antenna module. Therefore, according to a preferred embodiment of the present invention, a region designated by a may have a gap of 20 cm or more, so that the UHF RFID reader located on an upper side may recognize the UHF RFID tag without omission.

Meanwhile, the UHF RFID reader may be configured to identify UUID information of the UHF RFID tag when the UHF RFID tag is present inside the storage even when the user does not bring the UHF RFID tag close to the UHF RFID reader.

In other words, the information on the dental article may be monitored even when the medical staffs performs their tasks as they normally do without performing an additional action for allowing the reader to recognize the tag.

The UHF RFID reader may be configured to transmit information of a plurality of UHF RFID tags that are present inside the storage module to the server system.

According to one embodiment of the present invention, the UHF RFID tag may include only a UUID of each article, and information of the UHF RFID tag may include the UUID. In this case, the detailed article information may be stored in the server system in connection with the UUID.

The detailed article information may include article name information of the dental article, article name detailed specification (size, etc.), and expiration date information of the dental article.

According to another embodiment of the present invention, the UHF RFID tag may include the detailed article information together with the UUID of each article.

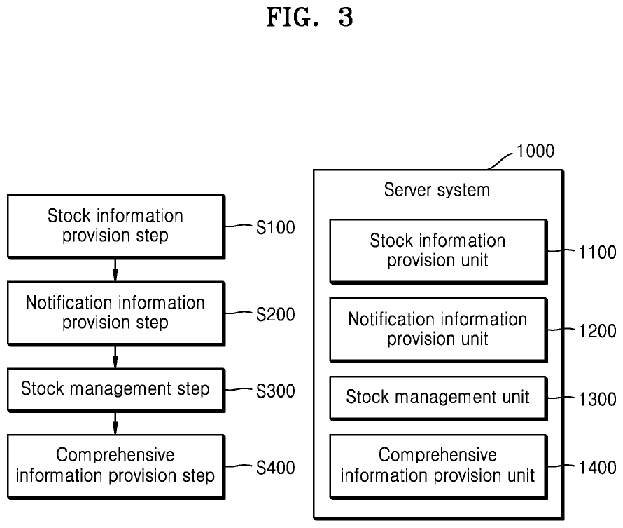

FIG. 3 schematically shows an operation and an internal configuration of a server system of the system for managing the dental article information according to one embodiment of the present invention.

According to a method for managing dental article information of one embodiment of the present invention, the server system may perform: a stock information provision step S100 of deriving current stock information for each dental article for each of a consultation room, a disinfection room, and an operating room based on the location information of the UHF RFID reader and the reading article information, and providing the current stock information to a user terminal; a notification information provision step S200 of setting safety stock information and expiration date information for each dental article based on information input from the user terminal, and generating notification information based on the current stock information, the safety stock information, and the expiration date information to provide the notification information to the user terminal; a stock management step S300 of providing the current stock information and the safety stock information for each dental article to the user terminal, and providing an order interface for each dental article to a user; and a comprehensive information provision step S400 of synthesizing partial results calculated in the stock information provision step and the notification information provision step to provide the synthesized partial results to the user terminal.

The server system may include a stock information provision unit 1100, a notification information provision unit 1200, a stock management unit 1300, and a comprehensive information provision unit 1400, which are configured to perform the stock information provision step S100, the notification information provision step S200, the stock management step S300, and the comprehensive information provision step S400, respectively.

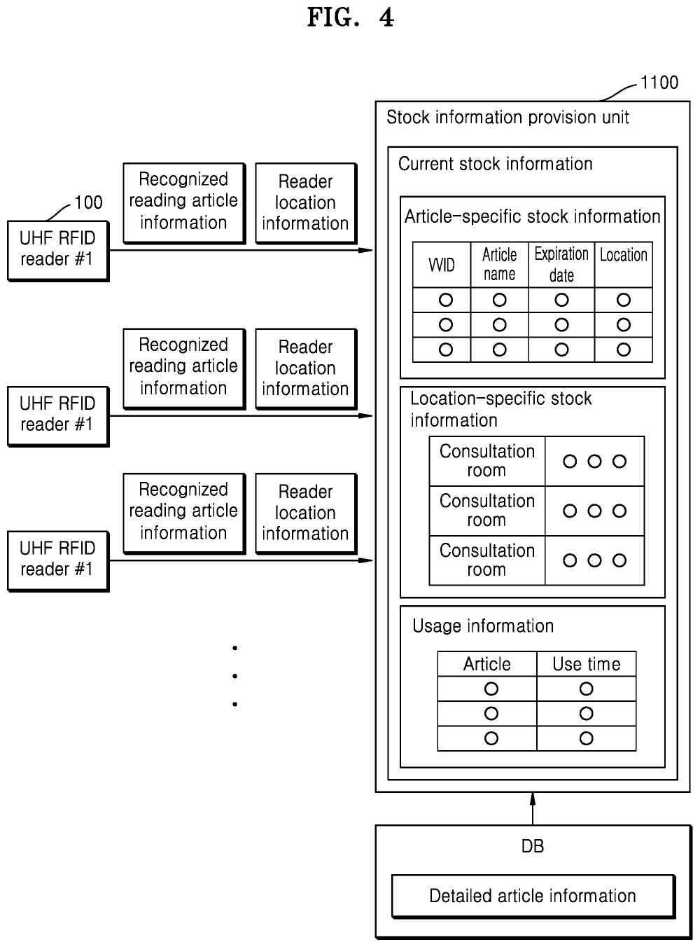

FIG. 4 schematically shows an operation of a stock information provision unit according to one embodiment of the present invention.

In FIG. 4, each of UHF RFID readers may transmit direct or indirect location information of the UHF RFID reader to the server system together with a plurality of pieces of reading article information recognized at a preset interval.

According to one embodiment of the present invention, the detailed article information may be stored in the server system for each individual dental article, and the detailed article information may include article name information of the dental article and expiration date information of the dental article. In this case, the pieces of the reading article information may include a UUID or a kind of identification information for the dental article, and the detailed article information may include connection information with the UUID and detailed information related to the article.

The detailed article information connected to the UUID may be received from a separate server outside the server system.

Alternatively, as described above, both the UUID and the detailed article information may be recorded in the reading article information.

Meanwhile, the stock information generated by the stock information provision unit may include article-specific stock information including a UUID, an article name, an expiration date, a location, and the like for each dental article.

The article-specific stock information may basically correspond to list-type information on all articles that are present in the storage module of the dental clinic.

Meanwhile, location-specific stock information may correspond to stock information of the dental article for each of the consultation room, the disinfection room, and the operating room, which is derived from the article-specific stock information.

In addition, based on the article-specific stock information, when a fluctuation occurs in an amount of the dental article that is present inside the storage module, usage information of the dental article may be derived based on the fluctuation, and the usage information may also be continuously updated in the server system.

According to one embodiment of the present invention, as a dental article having a specific UUID enters the storage module, the dental article may be registered with the article-specific stock information. When the dental article is recognized as going outside the storage module (an article that goes outside is excluded from the reading article information that is periodically received from the UHF RFID reader), and the dental article is recognized as not being brought into another storage module or the like even after a preset period, the dental article may be determined as a used dental article, so that the usage information may be determined.

Each of the dental articles and a use time (use date and time) of the dental article may be recorded in the usage information in the form of a list, and usage of a specific dental article for a preset period may be calculated from the usage information.

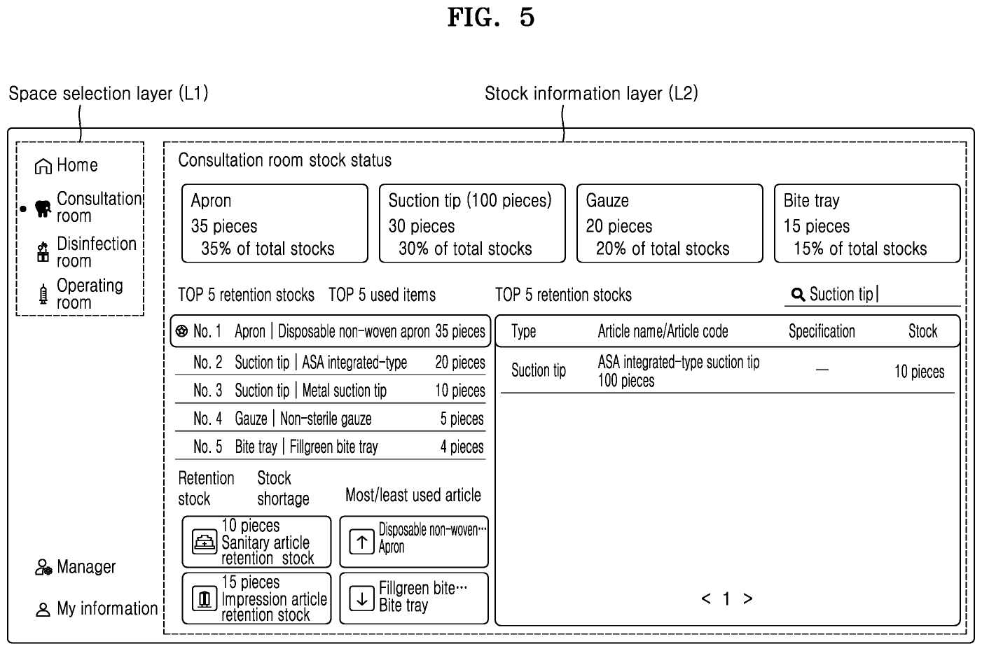

FIG. 5 schematically shows a screen of a user terminal displayed by a stock information provision step according to one embodiment of the present invention.

As shown in FIG. 5, a screen of the user terminal displayed by the stock information provision step may include: a space selection layer L1 for selecting one of the consultation room, the disinfection room, and the operating room; and a stock information layer L2 for displaying the current stock information of one of the consultation room, the disinfection room, and the operating room according to a space selected in the space selection layer.

The user may select a specific room in the space selection layer L1, so that a screen of the stock information layer on a right side, including a retention stock, a stock list, and the like, may be changed.

The stock information provision step may include: deriving information on a type and an amount of a dental article that is present inside the storage module at a preset interval based on information received from the UHF RFID reader; and deriving, when a fluctuation occurs in the amount of the dental article that is present inside the storage module, usage information of the dental article based on the fluctuation.

Meanwhile, the stock information layer may display: stock information of the dental article, which is sorted by a stock amount (corresponding to a ‘retention stock’ of FIG. 5); and a specific dental article extracted by applying a preset criterion to the usage information (corresponding to a ‘most/least used article’ of FIG. 5).

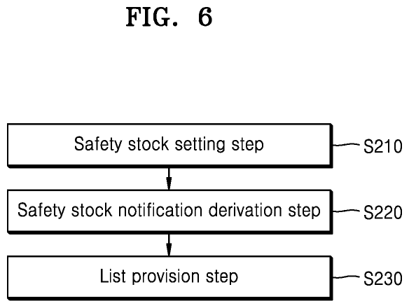

FIG. 6 schematically shows detailed processes of a notification information provision step according to one embodiment of the present invention.

The notification information provision step may include: a safety stock setting step S210 of providing an interface for setting a notification state for a safety stock and a safety stock amount for each dental article in the user terminal; a safety stock notification derivation step S220 of deriving safety stock notification information for a dental article in which a current stock amount for each dental article derived based on the current stock information is less than the safety stock amount; and a list provision step S230 of providing the safety stock notification information in a form of a list together with time information related to the safety stock notification information.

In the step S210, an interface for setting whether to provide a notification for each type of the dental article, and a stock amount upon the provision of the notification, that is, the safety stock amount may be provided. When an amount of the dental article of the type decreases to become less than or equal to the safety stock amount in the current stock information, the notification may be provided, and the step S210 may correspond to a step of performing setting for such a configuration.

In the step S220, the safety stock notification information may be generated for a dental article having an amount that is less than the safety stock amount set in the step S210.

Preferably, a generation interval of the safety stock notification information may vary based on a difference between the safety stock amount and the current stock amount for the dental article. According to one embodiment of the present invention, the safety stock notification information may be primarily generated when a stock amount of the dental article reaches the safety stock amount, and the safety stock notification information may be repeatedly generated according to a preset notification interval when the amount of the dental article does not exceed the safety stock amount after the primary generation. The notification interval may vary according to the difference between the safety stock amount and the current stock amount. In other words, as the difference between the safety stock amount and the current stock amount becomes greater, the notification interval may be automatically changed to become shorter.

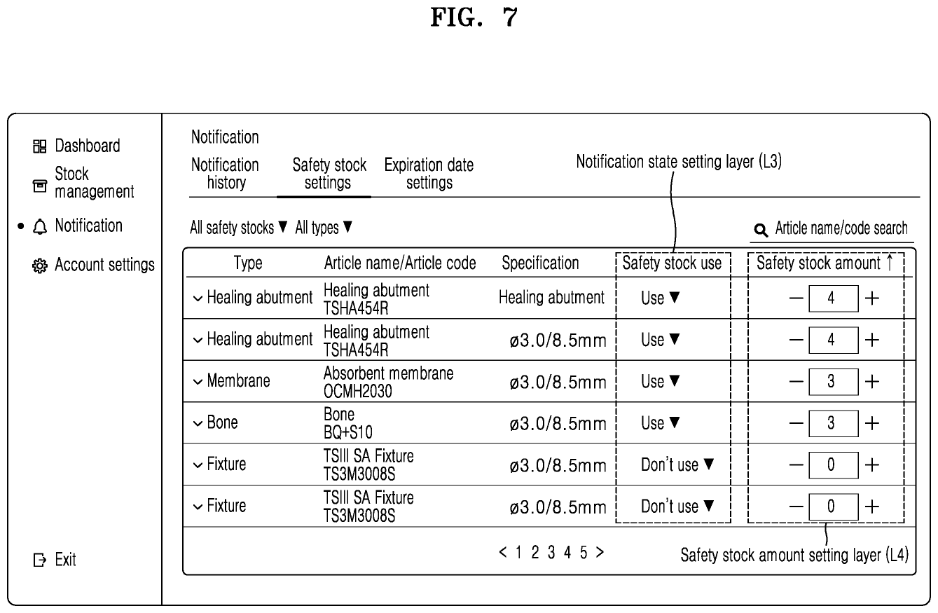

FIG. 7 schematically shows a screen of the user terminal for performing setting on a safety stock in the notification information provision step according to one embodiment of the present invention.

As shown in FIG. 7, it is possible to set whether to provide a notification for the safety stock for each type of the dental article through a manipulation in a notification state setting layer L3, and the safety stock amount may be set for each type of the dental article through a manipulation in a safety stock amount setting layer L4.

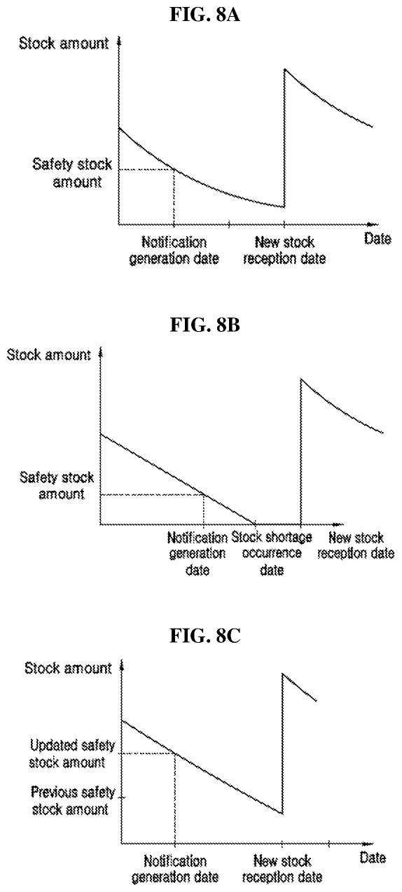

FIG. 8 schematically shows an automatic update process for the safety stock in the notification information provision step according to one embodiment of the present invention.

According to one embodiment of the present invention, the safety stock amount for each dental article may be set in the safety stock amount setting layer L4 as shown in FIG. 7.

Preferably, the safety stock amount setting layer L4 as shown in FIG. 7 may additionally include a tab that is referred to as “automatic safety stock update”, and the safety stock for the dental article may be automatically updated through a manipulation of such an automatic safety stock update tab.

FIG. 8A shows a normal case in which an amount of a specific dental article is decreased, a notification is generated when the amount of the specific dental article reaches the safety stock amount, and a new stock is subsequently received so that a stock amount is increased.

FIG. 8B shows a case in which an amount of a specific dental article is decreased, a notification is generated when the amount of the specific dental article reaches the safety stock amount, a stock shortage of the dental article subsequently occurs continuously before a new stock is received, and a stock amount is subsequently increased. In this case, the dental article may not be used between a stock shortage occurrence date and a new stock reception date. This corresponds to a case in which an order is delayed or a delivery/supply schedule is disrupted even when a safety stock notification is received.

According to one embodiment of the present invention, as shown in FIG. 8C, the safety stock amount may be automatically increased in a case of an article in which occurrence of a stock shortage is detected after a safety stock amount notification, so that the occurrence of the stock shortage may be prevented thereafter.

Preferably, an updated safety stock amount may be determined by a previous safety stock amount, a period between the stock shortage occurrence date and the new stock reception date, and daily usage of the dental article from a notification generation date to the stock shortage occurrence date.

According to one embodiment of the present invention, the updated safety stock amount may be determined by the following formula.
Updated safety stock amount=Previous safety stock amount+(Period between stock shortage occurrence date and new stock reception date * Daily usage of dental article from notification generation date to stock shortage occurrence date)

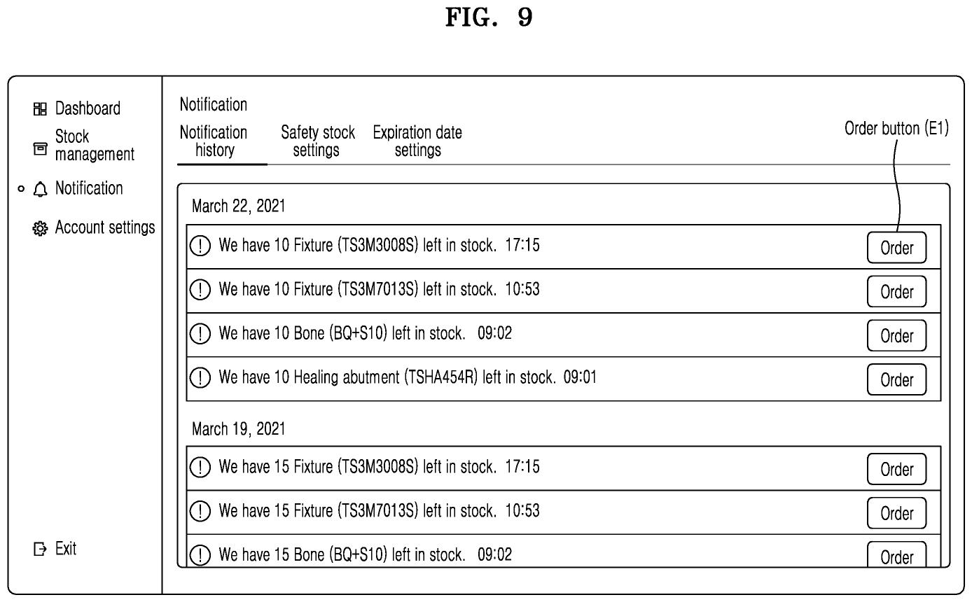

FIG. 9 schematically shows a screen of the user terminal for providing a notification list in the notification information provision step according to one embodiment of the present invention.

Although only a notification for the safety stock amount in the notification information provision step has been displayed in the screen shown in FIG. 9, information on a dental article having an expiration date that is imminent, which will be described below, may be displayed in the screen.

Preferably, an order button E1 may be displayed as shown in FIG. 9 in a notification list displayed in the notification information provision step, and the user may place an order for the dental article by simply clicking the order button E1.

According to one embodiment of the present invention, the server system may be connected to an external server for placing an order for the dental article, and the dental article may be automatically ordered according to an input of the order button E1 (and a subsequent input of the user to the order interface).

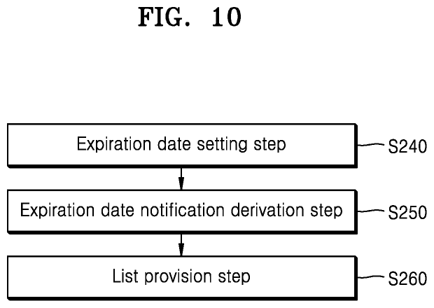

FIG. 10 schematically shows detailed processes of the notification information provision step according to one embodiment of the present invention.

As described above, the detailed article information may be stored in information recorded in the UHF RFID tag or the server system, and the detailed article information may include the expiration date information for each dental article.

According to one embodiment of the present invention, additionally or independently of the steps as shown in FIG. 6 described above, the notification information provision step may include: an expiration date setting step S240 of providing an interface for setting a reference expiration date for all dental articles in the user terminal; an expiration date notification derivation step S250 of deriving expiration date notification information for a dental article in which an expiration date has reached the reference expiration date among the dental articles; and a list provision step S260 of providing the expiration date notification information in a form list together with time information related to the expiration date notification information.

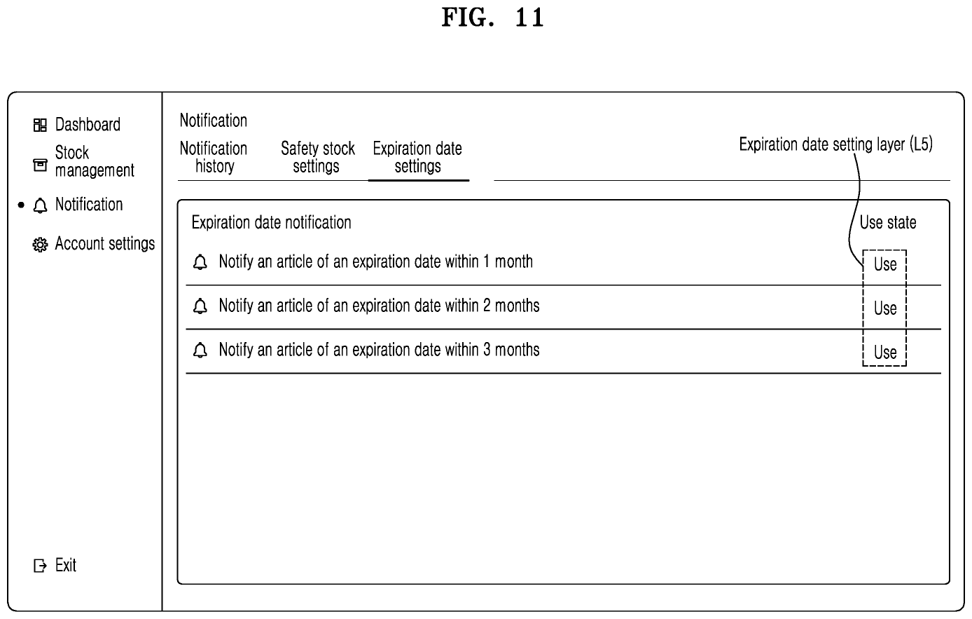

FIG. 11 schematically shows a screen of the user terminal for performing setting on an expiration date in the notification information provision step according to one embodiment of the present invention.

As shown in FIG. 11, the user may set whether to provide a notification for each expiration date.

For example, in a case where an expiration date of a specific dental article is 2021.7.4, when “Notify an article of an expiration date within 3 months” is selected, a notification for the dental article will be provided on 2021.4.4. Through such a configuration, the medical staff of the dental clinic may preferentially use a dental article in which a notification for an expiration date has been recognized if possible, so that a loss caused when the expiration date is passed may be prevented.

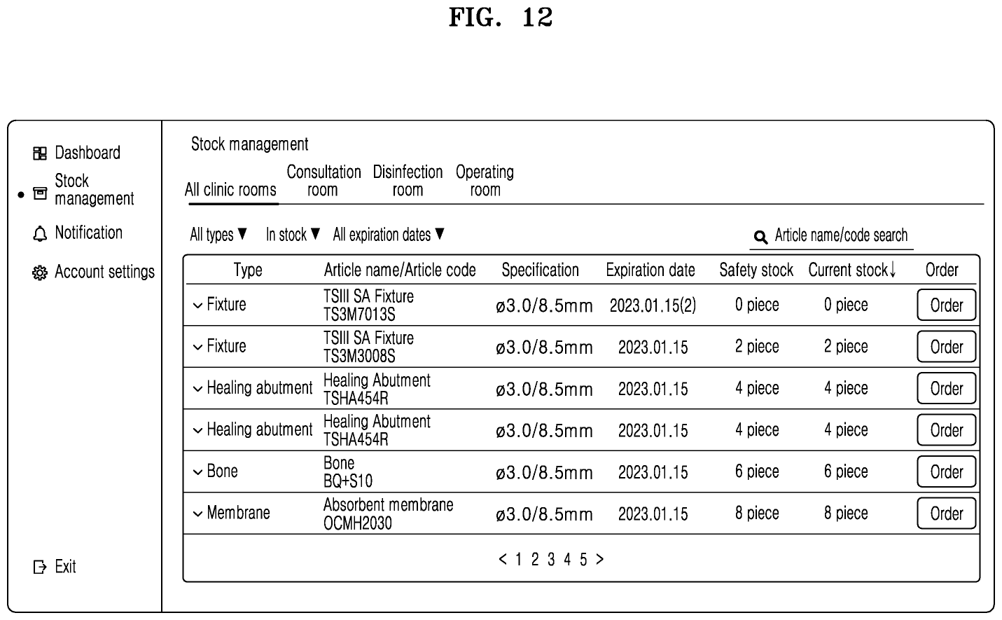

FIG. 12 schematically shows a screen of the user terminal in a stock management step according to one embodiment of the present invention.

In the stock management step, the current stock information and the safety stock information for each dental article may be provided to the user terminal, and an order interface for each dental article may be provided to the user.

As shown in FIG. 12, the user may select a stock management target room. In detail, when one of the consultation room, the disinfection room, and the operating room is selected, as shown in FIG. 12, a safety stock and a current stock may be displayed for each type of dental articles located inside the storage module of each of the rooms. In this case, the user may order the dental article directly from a stock management interface by inputting the order button.

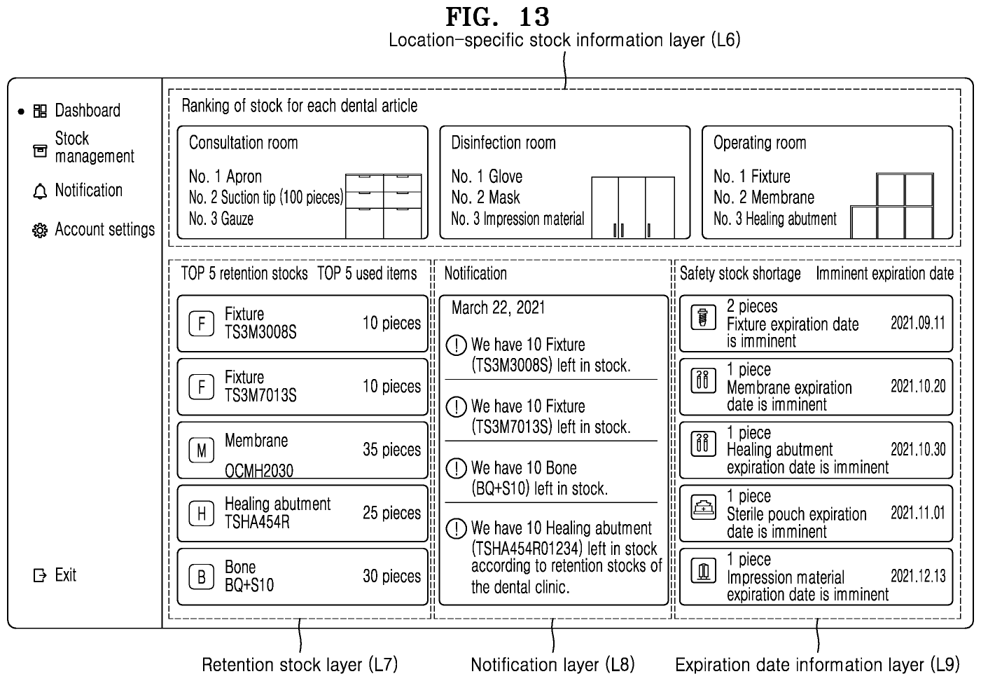

FIG. 13 schematically shows a screen of the user terminal in a comprehensive information provision step according to one embodiment of the present invention.

In the comprehensive information provision step, a screen through which the medical staff may collectively recognize the information generated in the stock information provision step and the notification information provision step may be provided.

As shown in FIG. 13, a screen of the user terminal displayed by the comprehensive information provision step may include: a location-specific stock information layer for displaying stock information of the dental article, which is sorted by a retention amount for each location; a usage stock layer L7 in which usage for a preset period in an entire dental clinic is sorted; a notification layer L8 in which recent notifications are displayed; and an expiration date information layer L9 for displaying dental articles having expiration dates that are imminent.

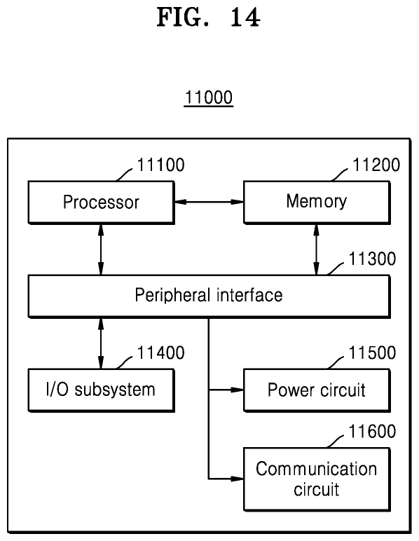

FIG. 14 schematically shows an internal configuration of a computing system such as the server system according to one embodiment of the present invention.

As shown in FIG. 14, a computing device 11000 may at least include at least one processor 11100, a memory 11200, a peripheral interface 11300, an input/output (I/O) subsystem 11400, a power circuit 11500, and a communication circuit 11600. In this case, an internal configuration of the computing device 11000 may be included in a computing system shown in FIG. 1, or the computing device 11000 may correspond to one embodiment of the computing system shown in FIG. 1.

The memory 11200 may include, for example, a high-speed random access memory, a magnetic disk, an SRAM, a DRAM, a ROM, a flash memory, and a non-volatile memory. The memory 11200 may include a software module, an instruction set, or other various data required for an operation of the computing device 11000.

In this case, access to the memory 11200 from other components of the processor 11100, the peripheral interface 11300, or the like may be controlled by the processor 11100.

The peripheral interface 11300 may couple an input and/or output peripheral device of the computing device 11000 to the processor 11100 and the memory 11200. The processor 11100 may execute the software module or the instruction set stored in the memory 11200 to perform various functions for the computing device 11000 and process data.

The I/O subsystem may couple various input/output peripheral devices to the peripheral interface 11300. For example, the I/O subsystem may include a controller for coupling the peripheral device, such as a monitor, a keyboard, a mouse, a printer, or a touch screen or a sensor if necessary, to the peripheral interface 11300. According to another aspect, input/output peripheral devices may be coupled to the peripheral interface 11300 without passing through the I/O subsystem.

The power circuit 11500 may supply a power to all or some of the components of the terminal. For example, the power circuit 11500 may include a power management system, at least one power source such as a battery or an alternating current (AC), a charging system, a power failure detection circuit, a power converter or inverter, a power status indicator, and any other components for generating, managing, or distributing a power.

The communication circuit 11600 may use at least one external port to enable communication with other computing devices.

Alternatively, as described above, the communication circuit 11600 may include an RF circuit, if necessary, to transmit and receive an RF signal, also known as an electromagnetic signal, thereby enabling the communication with other computing devices.

The above embodiment of FIG. 14 is merely an example of the computing device 11000, and a computing device 11000 may have a configuration or arrangement that omits some of the components shown in FIG. 14, further includes an additional component that is not shown in FIG. 14, or combines at least two components. For example, a computing device for a communication terminal in a mobile environment may further include a touch screen, a sensor, and the like in addition to the components shown in FIG. 14, and the communication circuit 11600 may include a circuit for RF communication in various communication schemes (such as Wi-Fi, 3G, LTE, Bluetooth, NFC, and Zigbee). The components that may be included in the computing device 11000 may be implemented as hardware, software, or a combination of both hardware and software including at least one integrated circuit specialized in signal processing or an application.

The methods according to the embodiment of the present invention may be implemented in the form of program instructions that may be executed through various computing devices, and may be recorded in a computer-readable medium. In particular, a program according to the present embodiment may be configured as a PC-based program or an application dedicated to a mobile terminal. An application to which the present invention is applied may be installed in the computing device 11000 through a file provided by a file distribution system. For example, the file distribution system may include a file transmission unit (not shown) for transmitting the file according to a request from the computing device 11000.

The device described above may be implemented as a hardware component, a software component, and/or a combination of the hardware component and the software component. For example, the devices and components described in the embodiments may be implemented by using at least one general purpose computer or special purpose computer such as a processor, a controller, an arithmetic logic unit (ALU), a digital signal processor, a microcomputer, a field programmable gate array (FPGA), a programmable logic unit (PLU), a microprocessor, or any other device capable of executing and responding to an instruction. The processing device may execute an operating system (OS) and at least one software application executed on the operating system. In addition, the processing device may access, store, manipulate, process, and generate data in response to the execution of the software. In some cases, one processing device is described as being used for convenience of understanding. However, it will be appreciated by those skilled in the art that the processing device may include a plurality of processing elements and/or a plurality of types of processing elements. For example, the processing device may include a plurality of processors or one processor, and one controller. In addition, other processing configurations such as a parallel processor may be possible.

The software may include a computer program, a code, an instruction, or a combination of at least one thereof, and may configure the processing device to operate as desired or instruct the processing device independently or collectively. In order for the software and/or data to be interpreted by the processing device or to provide an instruction or data to the processing device, the software and/or data may be permanently or temporarily embodied in any type of machine, component, physical device, virtual equipment, computer storage medium or device, or transmitted signal wave. The software may be distributed over computing devices, which are connected through a network, so as to be stored or executed in a distributed manner. The software and data may be stored in at least one computer-readable recording medium.

The method according to the embodiment may be implemented in the form of a program instruction that may be executed through various computer mechanisms, and may be recorded in a computer-readable medium. The computer-readable medium may include a program instruction, a data file, a data structure, and the like, alone or in combination with each other. The program instruction recorded on the medium may be specially designed and configured for the embodiment, or may be known and available to those skilled in the art of computer software. An example of the computer-readable recording medium includes a magnetic media such as a hard disk, a floppy disk, and a magnetic tape, an optical media such as a CD-ROM and a DVD, a magneto-optical media such as a floptical disk, and a hardware device specially configured to store and execute a program instruction such as a ROM, a RAM, and a flash memory. An example of the program instruction includes a high-level language code that may be executed by a computer by using an interpreter or the like, as well as a machine language code generated by a compiler. The hardware device may be configured to operate as at least one software module to perform the operations of the embodiments, and vice versa.

Although the above description has been made with reference to specific embodiments and drawings, various modifications and changes can be made by those skilled in the art from the above description. For example, even when the described techniques are performed in an order that is different from the described manner, and/or the described components such as systems, structures, devices, and circuits are coupled or combined in a form that is different from the described manner, or replaced or substituted by other components or equivalents, appropriate results may be achieved. Therefore, other implementations, other embodiments, and equivalents to the claims are also within the scope of the appended claims.

What is claimed is:

1. A system for managing dental article information, the system comprising:
a UHF RFID tag attached to a dental article;
a storage module having a space in which the dental article is stored;
a UHF RFID reader disposed on an upper side of the storage module, and configured to read reading article information of the UHF RFID tag attached to the dental article stored in the storage module; and
a server system configured to receive the reading article information, and store location information of the UHF RFID reader and detailed article information corresponding to the reading article information,
wherein the server system is configured to perform:
a stock information provision step of deriving current stock information for each dental article for a consultation room, a disinfection room, and an operating room based on the location information of the UHF RFID reader and the reading article information, and providing the current stock information to a user terminal;
a notification information provision step of setting safety stock information and expiration date information for each dental article based on information input from the user terminal, and generating notification information based on the current stock information, the safety stock information, and the expiration date information to provide the notification information to the user terminal; and
a stock management step of providing the current stock information and the safety stock information for each dental article to the user terminal, and providing an order interface for each dental article to a user.

2. The system of claim 1, wherein the storage module includes:
a cabinet having a single inner reception space without an inner partition wall; and
a system box stacked inside the cabinet, and in which at least one dental article is stored,
an antenna module of the UHF RFID reader is arranged in a planar shape on a top surface of the cabinet, and
the antenna module of the UHF RFID reader and a top surface of the inner reception space of the cabinet are spaced apart from each other by 20 cm or more.

3. The system of claim 1, wherein the UHF RFID reader is configured to identify UUID information of the UHF RFID tag when the UHF RFID tag is present inside the storage even when the user does not bring the UHF RFID tag close to the UHF RFID reader, and
the UHF RFID reader is configured to transmit information of a plurality of UHF RFID tags that are present inside the storage module to the server system.

4. The system of claim 1, wherein the detailed article information is stored in the server system for each individual dental article, and
the detailed article information includes article name information of the dental article and the expiration date information of the dental article.

5. The system of claim 1, wherein a screen of the user terminal displayed by the stock information provision step includes:
a space selection layer for selecting one of the consultation room, the disinfection room, and the operating room; and
a stock information layer for displaying the current stock information of one of the consultation room, the disinfection room, and the operating room according to a space selected in the space selection layer.

6. The system of claim 5, wherein the stock information provision step includes:
deriving information on a type and an amount of a dental article that is present inside the storage module at a preset interval based on information received from the UHF RFID reader; and
deriving, when a fluctuation occurs in the amount of the dental article that is present inside the storage module, usage information of the dental article based on the fluctuation, and
the stock information layer displays:
stock information of the dental article, which is sorted by a stock amount; and
a specific dental article extracted by applying a preset criterion to the usage information.

7. The system of claim 1, wherein the notification information provision step includes:
a safety stock setting step of providing an interface for setting a notification state for a safety stock and a safety stock amount for each dental article in the user terminal;
a safety stock notification derivation step of deriving safety stock notification information for a dental article in which a current stock amount for each dental article derived based on the current stock information is less than the safety stock amount; and
a list provision step of providing the safety stock notification information in a form of a list together with time information related to the safety stock notification information.

8. The system of claim 1, wherein the detailed article information includes the expiration date information for each dental article, and
the notification information provision step includes:
an expiration date setting step of providing an interface for setting a reference expiration date for all dental articles in the user terminal;
an expiration date notification derivation step of deriving expiration date notification information for a dental article in which an expiration date has reached the reference expiration date among the dental articles; and
a list provision step of providing the expiration date notification information in a form list together with time information related to the expiration date notification information.

9. A method for managing dental article information by using a dental article management system, in which the dental article management system includes
a UHF RFID tag attached to a dental article,
a storage module having a space in which the dental article is stored,
a UHF RFID reader disposed on an upper side of the storage module, and configured to read reading article information of the UHF RFID tag attached to the dental article stored in the storage module, and
a server system configured to receive the reading article information, and store location information of the UHF RFID reader and detailed article information corresponding to the reading article information, the method comprising:
a stock information provision step of deriving, by the server system, current stock information for each dental article for a consultation room, a disinfection room, and an operating room based on the location information of the UHF RFID reader and the reading article information, and providing the current stock information to a user terminal;
a notification information provision step of setting, by the server system, safety stock information and expiration date information for each dental article based on information input from the user terminal, and generating notification information based on the current stock information, the safety stock information, and the expiration date information to provide the notification information to the user terminal; and
a stock management step of providing, by the server system, the current stock information and the safety stock information for each dental article to the user terminal, and providing an order interface for each dental article to a user.
